(19) United States
(12) Patent Application Publication
(10) Pub. No.: US 2025/0090826 A1
(43) Pub. Date: Mar. 20, 2025

(54) SYSTEMS AND METHOD FOR IMPROVED SURGICAL OR GRAFT ACCESS

(71) Applicant: ABIOMED, Inc., Danvers, MA (US)

(72) Inventors: Christopher Sheils, Danvers, MA (US); Dustin Seth West, Danvers, MA (US); Alexander Ship, Danvers, MA (US); James Gabriel, Danvers, MA (US); Margi Patel, Danvers, MA (US); Jeremy Nezaria, Danvers, MA (US); Christopher Theodore Bazdanes, Danvers, MA (US); Vincent Roy, Danvers, MA (US); Qingchao Kong, Danvers, MA (US)

(73) Assignee: ABIOMED, Inc., Danvers, MA (US)

(21) Appl. No.: 18/891,386

(22) Filed: Sep. 20, 2024

Related U.S. Application Data
(60) Provisional application No. 63/539,373, filed on Sep. 20, 2023.

(51) Int. Cl. A61M 39/06; A61F 2/06; A61M 39/02; A61M 60/13; A61M 60/216; A61M 60/865

(52) U.S. Cl. CPC A61M 39/06; A61F 2/06; A61M 60/13; A61M 60/216; A61M 60/865; A61M 2039/0258; A61M 2039/0264; A61M 2039/0273; A61M 2039/0297; A61M 2039/062; A61M 2039/064; A61M 2205/0238

(57) ABSTRACT

A graft system may be provided. The graft system may include a graft, a hemostasis valve operably coupled to a proximal end of the graft, and a clamp disposed around the proximal end of the graft and configured to hold the hemostasis valve in place relative to the graft. The graft may include a woven or knitted fabric. The clamp may be removably, or permanently, attached to the graft. The hemostasis valve may include a slit membrane disposed within a housing. The hemostasis valve may include an introducer sheath coupled to the housing. The housing may include two portions coupled together along two axial seams, each axial seam extending from a proximal end to a distal end of the hemostasis valve. A grommet may be coupled to an end of the graft.

### CROSS-REFERENCE TO RELATED APPLICATIONS

The present application claims priority to U.S. Provisional Patent Application No. 63/539,373, filed Sep. 20, 2024, the contents of which are incorporated by reference herein in its entirety.

### TECHNICAL FIELD

The present disclosure relates to techniques for improving surgical or graft access.

### BACKGROUND

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the IMPELLA® blood pumps. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

Catheter-based blood pumps, and specifically blood pumps intended to be inserted into a patient's heart, are traditionally surgically inserted or implanted into a blood vessel, such as a chamber of a patient's heart, using external imaging equipment. Typically, a guidewire may be inserted via an introducer sheath into, e.g., a femoral or jugular vein, and, typically under fluoroscopy or other similar imaging technique, the guidewire is steered through the patient's vasculature. The blood pump is then advanced along the guidewire, also under fluoroscopy, until the blood pump is in a desired location.

### BRIEF SUMMARY

In various aspects, a graft system may be provided. The graft system may include a graft, a hemostasis valve operably coupled to a proximal end of the graft, and a clamp disposed around the proximal end of the graft and configured to hold the hemostasis valve in place relative to the graft.

The graft may include a woven or knitted fabric. The graft may include a coating. The coating may include cellulose.

The clamp may be removably attached, or permanently attached, to the graft.

The hemostasis valve may include a slit membrane disposed within a housing. The hemostasis valve may include an introducer sheath coupled to the housing. The housing may include two portions (e.g., a first portion and a second portion) coupled together along two axial seams. Each axial seam may extend from a proximal end of the hemostasis valve to a distal end of the hemostasis valve. Each seam may be configured to provide a preferential breaking line for separating the hemostasis valve into the two portions. The housing may include at least two handles. The at least two handles may include a first handle on the first portion and a second handle on the second portion of the housing.

The graft system may include a grommet, where one end of the grommet is coupled to an end of the graft. The grommet may include one or more threads. The graft system may include at least one ring configured to be operably coupled to the grommet via the one or more threads. The grommet may include at least one inner flange. The grommet may include a coating. The coating may include cellulose.

A kit may be provided. The kit may include a graft, a hemostasis valve configured to be operably coupled to a proximal end of the graft, and a clamp configured to be disposed around the proximal end of the graft and hold the hemostasis valve in place relative to the graft. The kit may include a tool configured to cut the graft. The kit may include a Y-connector.

A device may be provided. The device may include a grommet, and a sheath having a distal end coupled to a proximal end of the grommet. A hemostasis valve may be coupled to a proximal end of the sheath. A cap may be configured to be operably coupled to a proximal end of the hemostasis valve and at least partially compress a membrane of the hemostasis valve. The grommet may include one or more threads. The device may include at least one ring configured to be operably coupled to the grommet via the one or more threads. The grommet may include at least one inner flange.

A stent device may be provided. The stent may include a stent portion (which may be, e.g,. a substantially tubular member), and a y-branch portion having a common arm and two side arms coupled to the common arm. The common arm may be coupled to an end of the stent portion.

A tool for cutting grafts may be provided. The tool may include a frame having two portions configured to receive a graft between the two portions. The tool may include a mandrel extending through at least one of the two portions. The mandrel configured to cut a graft placed between the two portions. A gap between the two portions may be adjustable. The tool may include a dial configured allow a user to select a size of a vessel in which the graft is to be placed. The tool may be configured to adjust a cut based on a setting of the dial.

A method for anastomosis may be provided. The method may include providing a graft system as disclosed herein, creating an opening in a vessel, and adhering (e.g., gluing) a graft of the graft system to the vessel. Adhering the graft to the vessel may include, e.g., providing an expanding o-ring and/or balloon, the expanding o-ring and/or balloon containing an adhesive coating, adhering the graft to the expanding o-ring and/or balloon, and adhering the expanding o-ring and/or balloon to the vessel such that the graft is between the expanding o-ring and/or balloon and the vessel.

### BRIEF DESCRIPTION OF DRAWINGS

FIG. 1 is an illustration of a graft system.

FIG. 2 is an illustration showing a prior art graft technique.

FIG. 3 is an illustration of an example access site, showing an axillary vein, axillary artery, and cephalic vein near the clavicle and various muscles.

FIGS. 4A-4C are illustrations showing three different woven fabric patterns for grafts, including a plain weave (4A), a twill weave (4B), and a satin weave (4C).

FIGS. 5A-5B are illustrations showing two different knitted fabric patterns, including a weft knit (5A) and a warp knit (5B).

FIG. 6 is a table showing a comparison of grafts vs bevel angles.

FIG. 7 is an illustration of a hemostasis valve.

FIGS. 8 and 9 are illustrations of cross-section views of various embodiments of a grommet.

FIG. 10 is an illustration of a grommet coupled to a sheath and housing of a hemostasis valve, with a cap for the hemostasis valve.

FIG. 11 is an illustration of a grommet coupled to a graft.

FIG. 12 is an illustration of a Y-connector.

FIG. 13 is an illustration of a tool.

FIG. 14 is an illustration of a graft system inserted partially into a vessel.

FIG. 15 is an illustration of a cross-sectional side view of a graft system inserted partially into a vessel.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

The accompanying drawings, which are incorporated in and constitute a part of this specification, illustrate embodiments of the present disclosure. It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

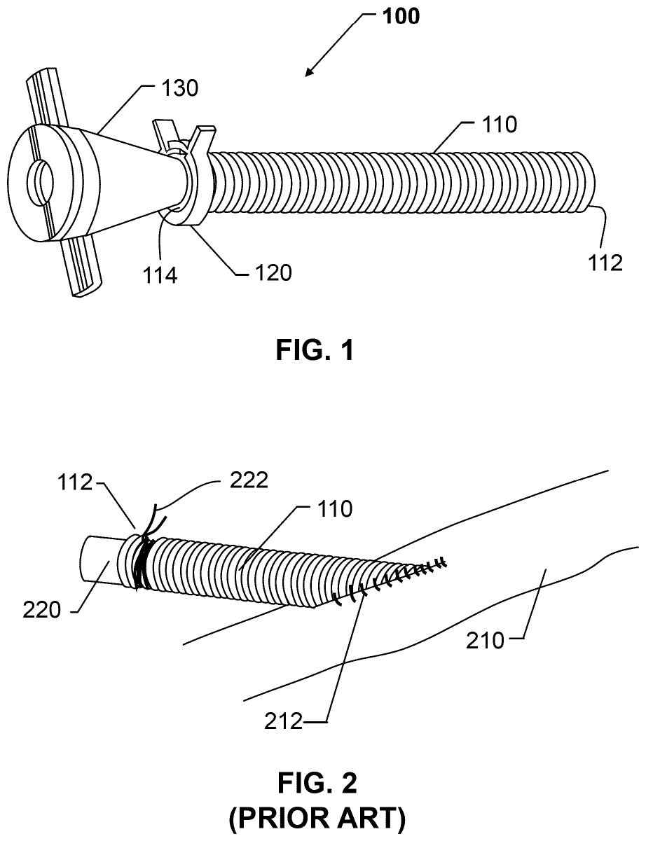

Referring to FIG. 1, a graft system (100) in accordance with the present disclosure is shown. The graft system (100) may include a graft (110) having a proximal end (114) and a distal end (112). The graft system may include a clamp (120). The graft system may include a hemostasis valve (130).

Conventionally, a graft (110) is typically sutured to a vessel. As shown in FIG. 2, a graft (110) may be secured to vessel (210) using surgical sutures (212). Such sutures (212) are common medical products available in many different shapes, sizes and materials. Such grafts (e.g., graft (110)) are typically used in medical procedures where a medical professional needs to gain access to a vessel (e.g., vessel (210)) in order to introduce and/or remove a medical product and/or device, or to allow blood or bodily fluid to be circulated through the graft (110) to other parts of the vasculature or to the outside. In the case that the graft (110) is used to introduce a medical device (220), the proximal end (114) of the graft is typically at least partially occluded using a medical thread (222) wrapped around the graft (110) and knotted as shown in FIG. 2. However, securing a medical device (220) using a medical thread (222) is not very reproducible and leads to blood loss during the time it is implemented.

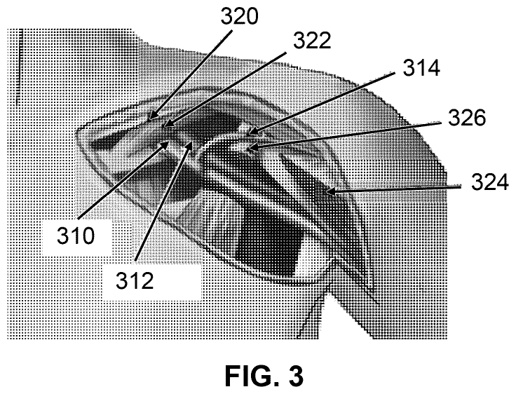

FIG. 3 shows an example of locations to which a graft could be introduced. For example, near the clavicle (320), the subclavius muscle (322), the divided pectoralis major (324) and minor (322) muscles, and cephalic vein (314), a graft could be introduced to an axillary vein (310) and/or an axillary artery (312). As seen in FIG. 3, the anatomy makes it challenging to do so using conventional techniques; the disclosed device may be beneficial in making this part of a procedure easier.

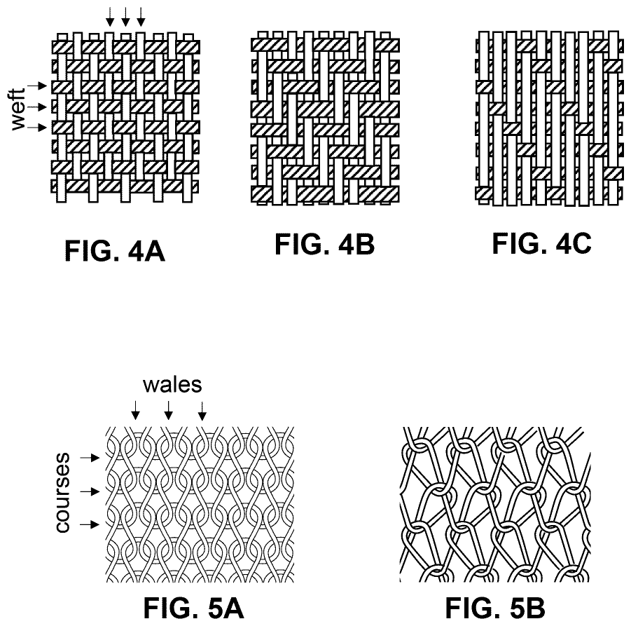

The graft (110) is typically a porous soft medical fabric, such as described in U.S. Pat. No. 3,953,566, and is intended for use as a conduit in contact with blood and biological tissue. Such a graft (110) is a commonly used medical product available in many different shapes, sizes and materials. For example, the graft may be a polytetrafluoroethylene (PTFE) or polyester (e.g., DACRON® polyester) graft. In some cases, referring to FIGS. 4A-4C, the graft may be a woven fabric, which may be relatively non-porous (e.g., not intentionally porous), and may consist of warp and weft fibers disposed in various configurations, including a plain weave (4A), twill weave (4B), or satin weave (4C). Referring to FIGS. 5A-5B, the graft may be a relatively porous knitted fabric, having different courses and wales defining the knit pattern, including a weft knit (5A) or warp knit (5B), having clear porous portions of the knitted pattern.

For distal anastomosis one can use, e.g., a continuous 7-0 or 8-0 polypropylene suture with known techniques, such as the parachuting technique. It is essential that the sutures be placed symmetrically and with even spacing to ensure adequate hemostasis, and that the grafts be handled with care to avoid endothelial injury.

An example conventional distal anastomosis technique can be described as follows. First, various sutures must be placed. A suture may be placed on the graft from outside to inside at a position that is two stitches away from the heel (visualized as a clock face, the heel of the graft being at the 6 o'clock position, lining up with the 12 o'clock position on the artery). This suture would be placed at the 4 o'clock position on the graft. Then, a stitch is taken on the coronary artery from inside the arteriotomy to outside at a position that is two stitches away from the heel (e.g., 2 o'clock position on the artery), then a stitch is then taken from outside to inside on the graft at a position one stitch away from the heel, and then inside to outside in the artery at a position one stitch away from the heel (e.g., 1 o'clock position on the artery). This process may be repeated for various amounts of additional stitches (e.g., 1 or more additional stitches). Next, the graft is parachuted down onto the coronary artery. The suture is then run in a counterclockwise direction from outside to inside through the graft and inside to outside through the coronary artery toward the toe. Particular care must be taken around the toe of the anastomosis to ensure that the suture does not back-wall or stenose the artery. Once around the toe, the running suture is continued in a counterclockwise direction from outside to inside through the graft and inside to outside through the coronary artery toward the initial suture. Once the initial suture tail is reached, the needles may be cut and the suture is tied down.

Similarly, conventional proximal anastomosis joins the venous or arterial graft with the aorta. As is understood in the art, appropriate sutures (such as a 5-0 polypropylene suture) with the parachuting technique may be used. Just as with distal anastomosis, it is essential that the sutures be placed symmetrically and with even spacing to ensure adequate hemostasis, and that the grafts be handled with care to avoid endothelial injury.

An example conventional proximal anastomosis technique can be described as follows. First, a small punch aortotomy may be made in the aorta. Second, sutures may be placed. A suture may be placed through the graft from outside to inside at a position that is 2 spaces away from the heel (e.g., visualized as a clock face, this is the 4 o'clock position on the graft). A stitch may be taken on the aorta from inside the aortotomy to outside at a position that is 2 spaces away from the heel (e.g., 2 o'clock position on the aorta), then taken from outside to inside through the graft at a position one space away from the heel (e.g., 5 o'clock position on the graft) and then inside to outside through the aorta at a position one space away from the heel (e.g., 1 o'clock position on the aorta). This process is repeated for 5 stitches. Just as with the example distal anastomosis technique, the graft may be parachuted down onto the aorta, and the suture may then be run in a counterclockwise direction from outside to inside through the graft and inside to outside through the aorta toward the toe of the anastomosis. Once the toe has been reached (6 o'clock position), the suture tail may be dropped and secured with a rubber-shod clamp. The other tail of the suture is then run in a clockwise direction from outside to inside through the aorta and inside to outside through the graft toward the toe. Once the initial suture tail is reached, the needles may be cut and the suture tied down.

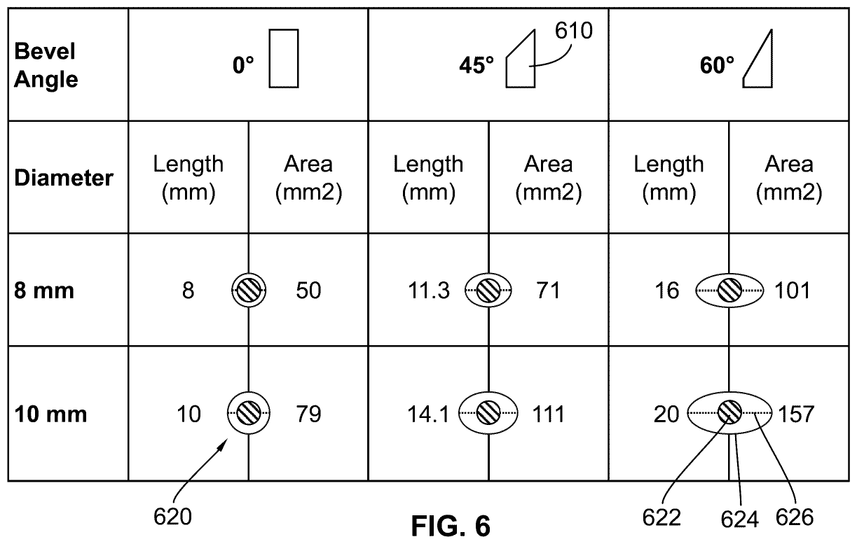

Referring to FIG. 6, for anastomosis, the relative size of a graft (622) versus a bevel (610) can be seen. Specifically, for bevel angle (0°, 45°, and 60°), and for each diameter (8 mm and 10 mm) in FIG. 6, an image (620) is shown, showing the relative size of the graft (622) for that bevel angle with its relative length (624), and a 5.5 mm hole (626) over which the graft could be placed, for comparison. As can be seen, for the same bevel, a 10 mm graft anastomosis is 56% larger than an 8 mm, and an 8 mm graft requires more than a 45-degree bevel to have an area larger than a 10 mm unbeveled graft.

Referring back to FIG. 1, the graft system (100) shows the use of the hemostasis valve (130) and a clamp (120) to allow the introduction of a medical device without the risk of blood loss. Specifically, the hemostasis valve (130) may be coupled to the graft (110) at the proximal end (114) of the graft. The clamp (120) is disposed around a portion of the graft (110) and a portion of the hemostasis valve (130) at or near the proximal end (114) of the graft The clamp (120) may eliminate the need to use a medical thread (222) and eliminates the subjectivity of the procedure, leading to immediate hemostasis and a more reproducible outcome.

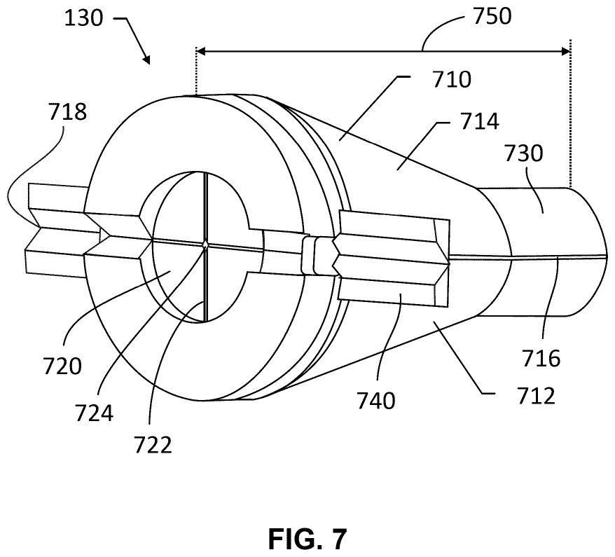

Referring to FIG. 7, an embodiment of the hemostasis valve (130) in accordance with the present disclosure is shown. The hemostasis valve (130) may include a housing (710). The hemostasis valve (130) may include a slit membrane (720), where slits (722) allow, e.g., a medical device to pass through a center (724) of the slit membrane (724). The hemostasis valve (130) may include an introducer sheath (730).

The housing (710) may include two distinct portions (e.g., two distinct halves, although an exact 50/50 split may be present but is not required). The two distinct portions may include a first portion (712) and a second portion (714). In certain aspects, the two portions may advantageously be joined or coupled by two seams (716, 618) that travel the entire length (750) of the hemostasis valve (130). The seams (716, 618) are intended to, e.g., thin the wall of the hemostasis valve (130) to provide a preferential breaking line in the event that the hemostasis valve (130) is split into the two portions (e.g., first portion (712) and second portion (714)).

Handles (740) are intended to assist the user in handling the hemostasis valve (130) as well as providing a handle to assist in separating the hemostasis valve (130) into the two separate portions (e.g., first portion (712) and second portion (714)).

In certain aspects, the seams (716, 618) may travel the middle of the handles (740) in order to facilitate breaking the handles (740) into two parts (e.g., one part for first portion (712), one part for second portion (714)).

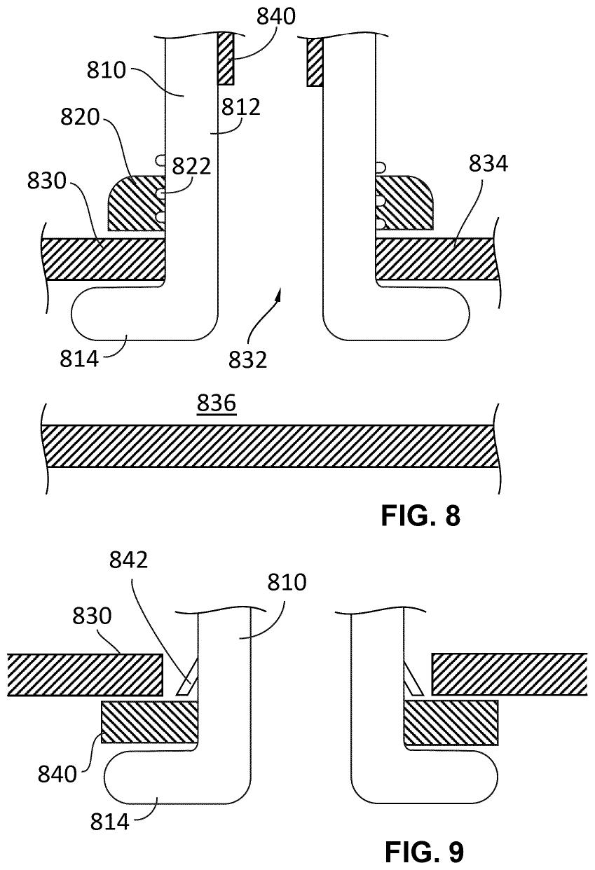

Referring to FIG. 8, in certain aspects, a single-use multi-tool may be used to punch a hole (832) through a wall (834) of a vessel (830), to allow access to an interior (836) portion of the vessel, and then staple a hard grommet (810) to the vessel, making an access hole.

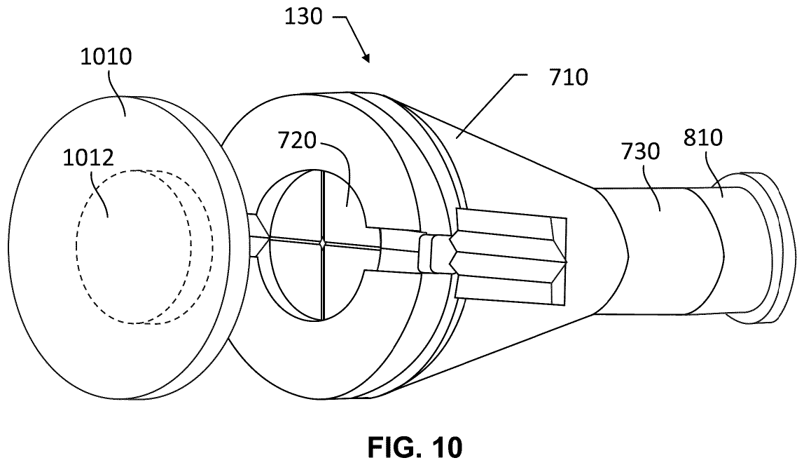

The single-use multi tool may function similar to how a grommet tool functions. First the device may have a portion that punctures and goes into the vessel and opens up—this may utilize “J-shaped” hooks, similar to what is shown as forming the distal portion (814) in FIGS. 8-9. There may be, e.g., a ring portion with threads that would snap onto the band that opens up. This tool would be used to further open up the access hole from more than a puncture, and to maintain hemostatic without having to do sutures.

In FIG. 8, the cross-section of the grommet (810) may be seen, where the grommet (810) may have proximal portion (812) (or proximal end) and a distal portion (814) (or distal end). The distal portion (814) may be configured to be disposed within the interior portion (836) of the vessel, and may be grommet may be configured to be disposed at or near an interior surface of the vessel. The grommet may have threads (822) and a ring (820) deployed on top (e.g., external to the vessel) to secure the grommet to the vessel.

Referring to FIG. 9, the grommet may have inner flanges (842) to secure the grommet to the vessel. For example, the grommet may be configured to use a snap on band (840) in combination with the inner flanges (842) to hold the grommet in place. In various aspects, a small slit may be cut in the vessel, the grommet may be rolled up and passed through the small slit, and then a separate device may “un-roll” the grommet, such that the grommet is secured to the vessel by the inner flanges (see snap on bands below.

In some embodiments, a sheath (850) (which may be, e.g., an introducer sheath such as introducer sheath (730)) may be coupled to the grommet (e.g., to a proximal portion (812) (or proximal end) of the grommet. In such embodiments, no graft is required to be attached to the vessel. The sheath may include a hemostasis valve (e.g., hemostasis valve (130)) at a proximal end of the sheath (850)

In some embodiments, the hemostasis valve may heal/clot over to maintain hemostasis.

Referring to FIG. 10, in some aspects, the hemostasis valve (130) may include a cap (1010) that is configured to be disposed over the hemostasis valve (130) (e.g., at a proximal end of the housing (710) of the hemostasis valve (130)) and may compress the a membrane of the hemostasis valve (e.g., a portion (1012) of the cap (1010) may be configured to contact and compress the slit membrane (720)). Such an arrangement advantageously results in true hemostasis between the hemostatic valve and the cap. As will be understood, an advantage of including a hemostatic valve at the vessel is surgeons would know when there is bleedback.

If a graft is utilized, the device may be configured to also simultaneously tighten the loops on the opposing ends of the grafts as the hole is being punched. In this manner, instead of doing one-by-one sutures, the device may tighten everything all at once.

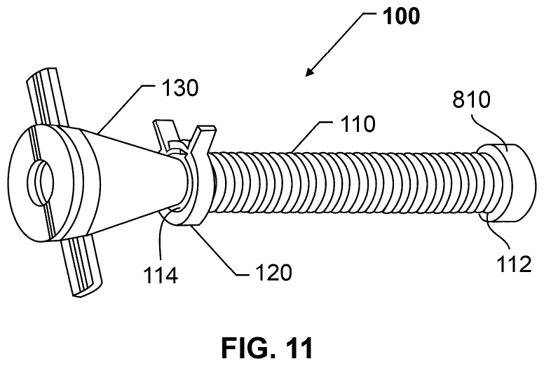

Referring to FIG. 11, in some embodiments, the grommet may be pre-attached to the graft (110). The graft and/or grommet may optionally include a collagen coating.

In various aspects, a method for end-to-end anastomosis may be provided. The method may include having a medical professional cut a vessel (such as an axillary artery or aorta) and then places a Y-connector that is provided with the pump insertion kit—this is effective achieving end-to-end anastomosis. The Y-connector may include a loop (or cuff) on each end to allow the surgeon to cuff the connector to the vessel. In various aspects, the connector may have a collagen coating or some other suitable coating. The collagen may allow the subject to fully maintain hemostasis.

An insertion kit may include different sizes and/or connectors with different angles in order to accommodate various patient anatomy. Providing these connectors may allow the sizes/angles to be fixed/predetermined and more consistent (rather than the medical professional to cut grafts manually. Such manual cutting results typically vary in size, length, angle, etc.

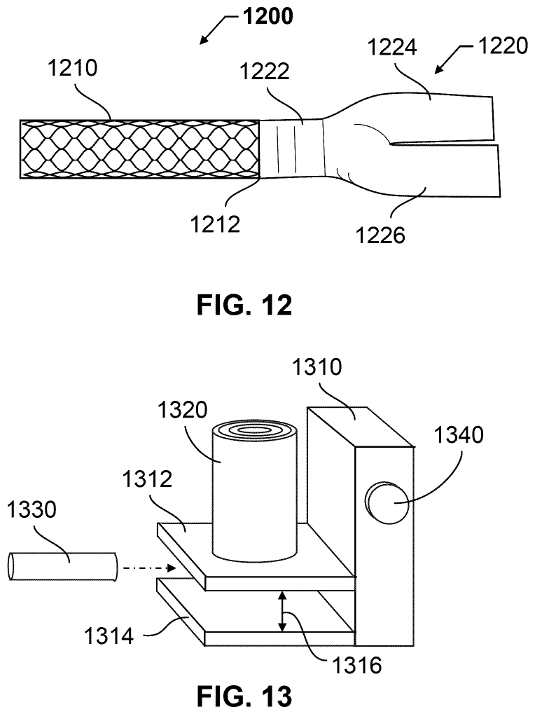

Referring to FIG. 12, The Y-connector (1200) may be a stent portion (1210) coupled to a Y-branch portion (1220) at one end (1212) of the stent portion (e.g., the stent portion (1210) may be coupled to a common arm (1222) of the Y-branch portion, with two side arms (1224, 1226) extending off of the common arm (1222). The two arms may form an internal channel that is operably coupled to a channel extending through the stent portion, such that a device passing through one side arm can extend through the common arm, then through the stent and pass out through a far end of the stent.

The stent may be, e.g., an expanded polytetrafluoroethylene (ePTFE) coated stent or any other suitable stent. The stent may also help keep the vessel open during support and when the patient is moving their arms. The Y-connector and/or the stent portion may be resorbable (e.g., may be composed of a resorbable material). In some aspects, at the end of a period of support, the Y-branch portion (1220) may be removed and the stent portion (1210) may remain in the vessel.

The grafts disclosed herein may be coupled to the vessel in various manners. In some aspects, the grafts may be glued to the vessel. For example, in some aspects, an expanding o-ring and/or balloon may be attached to the vessel. The expanding o-ring and/or balloon may have collagens/other coatings and/or glues on an external surface. This approach may provide hemostasis. In some aspects, the expanding o-ring and/or balloon may be a flat circle, or may have a saddle shape to accommodate vessel geometry. The graft may be glued to the expanding o-ring and/or balloon. The expanding o-ring and/or balloon may be used to seal at least one edge between the graft and the vessel.

In some aspects, use of the grafts disclosed herein may include pre-suturing the grafts and/or vessel. In some aspects, the grafts may be provided in a pre-sutured configuration. In some aspects, a device may be provided that starts the needle for suturing with the vessel—this will allow the medical professional to save time/steps with starting with the vessel. This overall approach may allow for the vessel to be pre-sutured and then the graft can be easily attached. The device, such as a stapler, may staple a vessel in an annular array. This may include utilizing annular rings that are collagen coated to allow the body to fully maintain hemostasis.

In certain aspects, a tool to cut a graft may be provided. Referring to FIG. 13, the tool may include a frame (1310) that may include one or more portions (1312, 1314) between which a graft (1330) may be inserted between. A mandrel (1320) may then extend through a portion of the frame (here, portion (1312)) and cut the graft when it was inserted between the two portions (1312, 1314). In some embodiments, the two portions may be arranged to be substantially planar in a direction orthogonal to a central axis of the mandrel (1320). In some embodiments, the two portions may be arranged to be substantially planar, but at an angle (not a 90° angle), relative to the central axis of the mandrel (1320). In some embodiments, the angle of the two portions relative to the central axis of the mandrel may be constant. In some embodiments, the angle of the two portions relative to the central axis of the mandrel may be adjustable.

One or more of the portions (1312, 1314) may be adjustable such that a minimum gap (1316) or distance between the portions can be adjusted. In some aspects, that minimum gap (1316) or distance may be fixed. In some embodiments, the gap between the portions may be constant (e.g., each part of the first portion (1312) may be the same distance from second portion (1312) as every other part of first portion (1312). In some embodiments, the gap may vary (e.g., one part of the first portion (1312) may be closer to the second portion (1314) than a different part of first portion (1312).

The tool may be provided with an insertion kit. The tool may be configured with a certain geometry that achieves a controlled angle and/or certain geometry of the graft. The medical professional would slide the existing graft into the tool and punch the graft, resulting in a specific geometry/angle.

In some embodiments, the geometry may be a bevel. In some embodiments, the geometry may be an “S-shape”. In certain instances, an S-shape may be a more optimal choice for smaller vessels when the graft is smaller than the vessel.

The tool may have a dial (1340) that may be adjusted based on the patient's vessel size. For example, a user may turn the dial to select a target vessel size. The tool may have multiple concentric mandrels, and, depending on the target vessel size, a larger or smaller diameter mandrel may be utilized.

In some embodiments, the grafts of various geometries on one or both ends of the graft may be provided with the insertion kit.

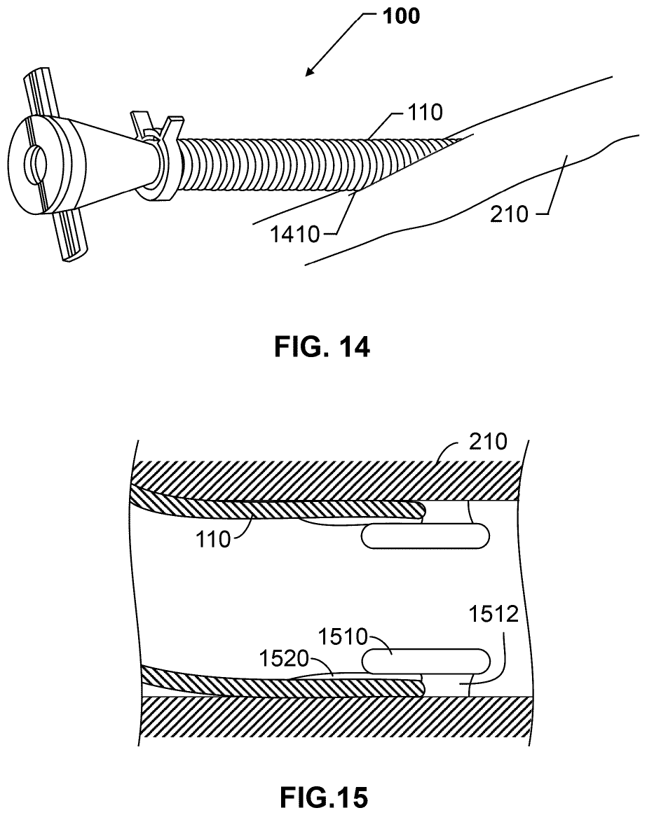

In various aspects, a method for anastomosis may be provided. The method may include, e.g., providing a graft system as disclosed herein, and creating an opening in a vessel via any appropriate technique. The method may then include adhering (e.g., via an adhesive, such as a glue) to a graft of the graft system to the vessel. This can be seen in FIG. 14, where a graft (110) of a graft system (100) has been introduced into a vessel (210) through an opening (1410). The graft may be, e.g., glued to an external surface, or an internal surface, of the vessel.

In some embodiments, gluing the graft to the vessel may include a multi-step process. The process may include providing an expanding o-ring and/or balloon (an expanding o-ring (1510) is shown in the cross-sectional view of FIG. 15). The expanding o-ring and/or balloon may include an adhesive coating (1512). The graft (110) may be adhered to the expanding o-ring and/or balloon (e.g., via an adhesive coating (1520) on the graft). The expanding o-ring and/or balloon may be adhered to the vessel (e.g., via adhesive coating (1512) such that the graft (110) is between the expanding o-ring (1510) and/or balloon and the vessel (210).

1. A graft system comprising:
a graft;
a hemostasis valve operably coupled to a proximal end of the graft; and
a clamp disposed around the proximal end of the graft and configured to hold the hemostasis valve in place relative to the graft.

2. The graft system of claim 1, wherein the graft comprises a woven or knitted fabric.

3. The graft system of claim 1, wherein the clamp is removably attached to the graft.

4. The graft system of claim 1, wherein the clamp is permanently attached to the graft.

5. The graft system of claim 1, wherein the hemostasis valve includes a slit membrane disposed within a housing.

6. The graft system of claim 5, wherein the hemostasis valve includes an introducer sheath coupled to the housing.

7. The graft system of claim 5, wherein the housing includes two portions, a first portion and a second portion, coupled together along two axial seams, each axial seam extending from a proximal end of the hemostasis valve to a distal end of the hemostasis valve.

8. The graft system of claim 7, wherein each seam is configured to provide a preferential breaking line for separating the hemostasis valve into the two portions.

9. The graft system of claim 7, wherein the housing includes at least two handles, the at least two handles including a first handle on the first portion and a second handle on the second portion of the housing.

10. The graft system of claim 1, wherein the graft includes a coating comprising cellulose.

11. The graft system of claim 1, further comprising a grommet, where one end of the grommet is coupled to an end of the graft.

12. The graft system of claim 11, wherein the grommet includes one or more threads.

13. The graft system of claim 12, further comprising at least one ring configured to be operably coupled to the grommet via the one or more threads.

14. (canceled)

15. (canceled)

16. A kit, comprising:
a graft;
a hemostasis valve configured to be operably coupled to a proximal end of the graft; and
a clamp configured to be disposed around the proximal end of the graft and hold the hemostasis valve in place relative to the graft.

17. The kit of claim 16, further comprising a tool configured to cut the graft.

18. The kit of claim 16, further comprising a Y-connector.

19. A device, comprising:
a grommet; and
a sheath having a distal end coupled to a proximal end of the grommet.

20-28. (canceled)

29. A method for anastomosis, comprising:
providing a graft system of claim 1;
creating an opening in a vessel; and
gluing a graft of the graft system to the vessel.

30. (canceled)
